(19) United States
(12) Patent Application Publication
(10) Pub. No.: US 2011/0040283 A1
(43) Pub. Date: Feb. 17, 2011

(54) Branch Vessel Targetter

(76) Inventor: Peter Lyon Harris, Hertfordshire (GB)

Correspondence Address: EPSTEIN & GERKEN, 1901 RESEARCH BOULEVARD, SUITE 340, ROCKVILLE, MD, 20850, US

(21) Appl. No.: 12/669,632

(22) PCT Filed: Jul. 16, 2008
(86) PCT No.: PCT/GB2008/002443

§ 371 Date: Aug. 2, 2010

(30) Foreign Application Priority Data
Jul. 20, 2007 (GB) 0714222.7

(51) Int. Cl. A61M 25/01

(52) U.S. Cl. 604/528

(57) ABSTRACT

Apparatus for positioning a catheter relative to a branch artery from a main artery comprising delivery means, a deployable catheter carrier having means for holding the catheter in a desired position within the main artery when delivered, and means for detecting the position of the catheter carrier.

This invention concerns a branch vessel targetter.

It is difficult to position a catheter relative to a branch vessel, such as a visceral artery, for delivery of, for example, a cutting catheter or wire.

An object of the invention is to provide a branch vessel targetter, such as for delivery of a catheter to a visceral artery via a main artery.

According to the invention there is provided apparatus for positioning a catheter relative to a branch artery from a main artery comprising delivery means, a deployable catheter carrier having means for holding the catheter in a desired position within the main artery when delivered and means for detecting the position of the catheter carrier.

The invention also provides a method of positioning a catheter relative to a side artery from a main artery comprising moving delivery means through the main artery to a desired position, the delivery means carrying a deployable catheter carrier having means for holding the catheter in a desired position within the main artery when delivered and means for detecting the position of the catheter carrier.

Preferably, the delivery means comprises a sheath for holding other components of the apparatus for delivery to a desired position within the main artery. The other components preferably include a nose cone that extends from the sheath.

The deployable catheter carrier preferably comprises an expandable component that can be delivered to the desired position in a collapsed state in the sheath before emerging from the sheath to expand to hold its position within the main artery by contacting the arterial wall.

The preferred expandable component is a cage. The cage preferably comprises a plurality of wires extending between a component axially movable relative to the nose cone and the nose cone. The nose cone is preferably on a rod and the movable component may be a sheath on the rod. When the axially movable component is moved away from the nose cone, the cage wires straighten so that the cage becomes narrow for deployment within the outer sheath. When the axially movable component is moved toward the nose cone the wires bend outwardly, once the outer sheath has been withdrawn to take a bulbous form, which can grip against the artery wall to hold the cage in position.

The catheter is preferably associated with a radio-opaque component and more preferably is linked to the cage by means of the radio-opaque component, whereby the position of the catheter carrier can be detected. Preferably the radio-opaque component is a ring. The ring is conveniently fixed to wires of the cage and preferably has inwards spokes to which the end of the catheter is joined.

The catheter is delivered to a desired position within the main artery using the radio opaque ring to detect the correct position, whereupon the outer sheath is withdrawn to uncover the expandable catheter carrier. The catheter carrier is then expanded to hold the catheter in position in the main artery, when the catheter can be used for delivery of a cutting catheter or wire to the side artery.

Preferably the catheter is sufficiently flexible to bend but sufficiently rigid so as not to deform too much.

This invention will now be further described, by way of example only, with reference to the accompanying drawings, in which:

FIG. 1 shows a visceral artery targetter according to the invention;

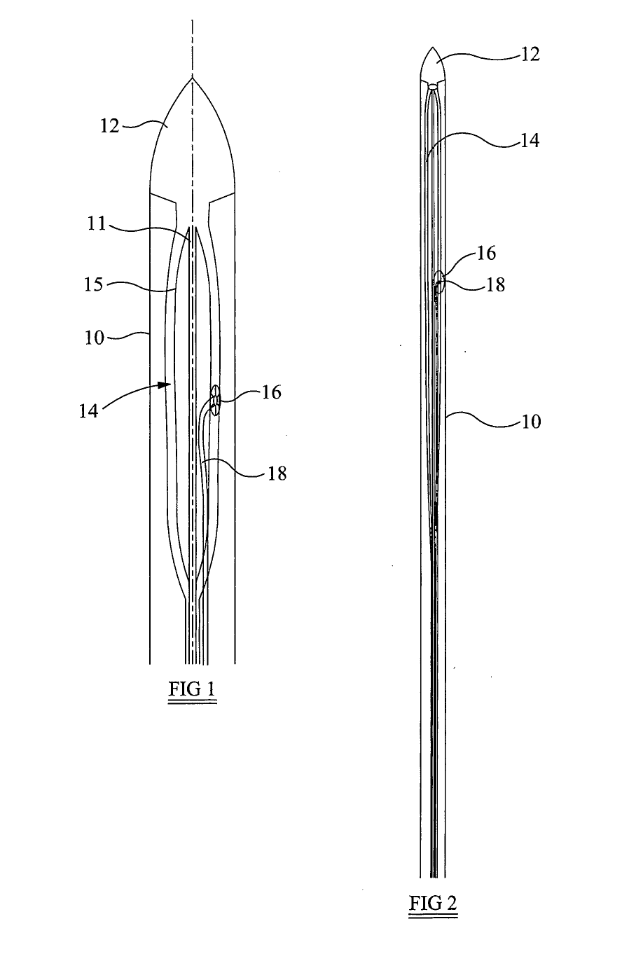

FIG. 2 is an enlarged view of the targetter of FIG. 1;

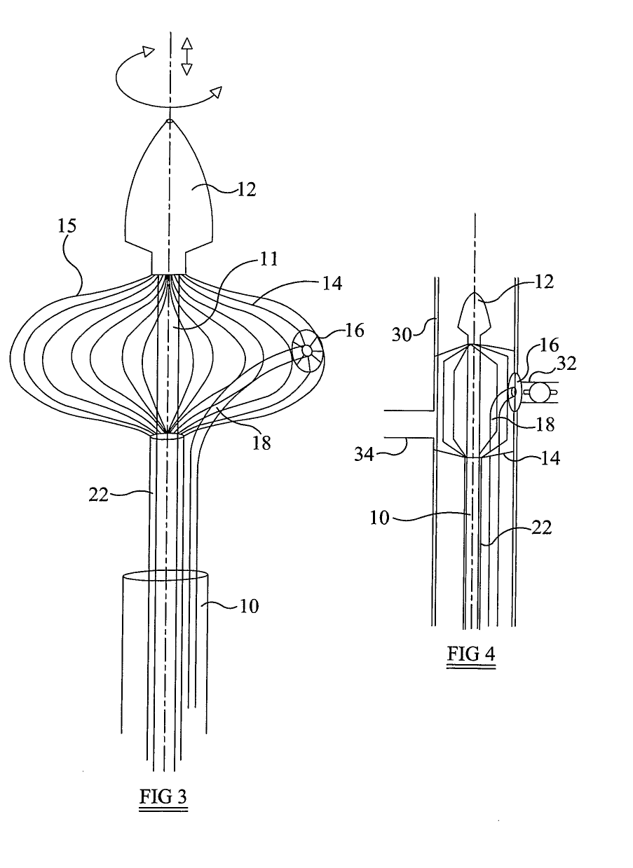

FIG. 3 shows the targetter of FIG. 1 opened; and

FIG. 4 shows the targetter deployed in an artery.

Referring to the accompanying drawings, a visceral artery targetter has an outer sheath 10 containing a rod 11 at the remote end of which is a nose cone 12. The rod 11 carries a second inner sheath 22 that is connected to the nose cone 12 by a wire cage 14. The wire cage 14 comprises radially arranged individual wires 15. The wires 15 are arranged, so that by sliding the inner sheath 22 away from the nose cone 12 straightens the wires 15 to fill a smaller sectional area and by sliding the inner sheath 22 towards the nose cone 12 causes the wires to bend and extend outwards to fill a wider sectional area. When expanded the cage is able to contact the wall of the main artery to hold the catheter 18 at a desired position. The cage 14 is further constrained by the outer sheath 10, so that with the wires 15 straightened, the outer sheath 10 can be slid up to the nose cone 12 to cover the cage for delivery of the whole through a main artery.

Also within the outer sheath 10 is a catheter sheath 18, which is connected between wires 15 by means of a radio-opaque ring 16.

The targetter is used to accurately position the catheter 18 in order, for example, to deploy a cutting catheter or wire through the catheter 18 for penetration into a visceral artery from the aorta, for example, in connection with deployment of a graft in a visceral artery to supplementing a graft in the aorta. When a graft is inserted into the aorta to support an aneurysm, the positioning of the graft can often overlap side arteries, such as visceral arteries, so blocking them. Also it is necessary to include in the visceral artery a graft to connect to the main arterial graft. However, due to the location of the visceral arteries accurately deploying a cutting catheter or wire is difficult.

By providing a radio opaque ring 16 around the catheter, it is possible to follow the positioning of the catheter by means of X-radiation. The targetter is delivered through a main artery 30 with the outer sheath 10 covering the cage. When the desired position for the catheter is reached, the sheath 10 is withdrawn and the inner sheath 22 slid over the rod 11 towards the nose cone in order to expand the cage to contact the artery wall to hold the targetter in position when the catheter will be bent towards the branch vessel 32 (see FIG. 4).

1. Apparatus for positioning a catheter relative to a branch artery from a main artery comprising delivery means, a deployable catheter carrier having means for holding the catheter in a desired position within the main artery when delivered and means for detecting the position of the catheter carrier.

2. Apparatus as claimed in claim 1, wherein the delivery means comprises a sheath for holding other components of the apparatus for delivery to a desired position within the main artery.

3. Apparatus as claimed in claim 2, wherein the other components include a nose cone that extends from the sheath.

4. Apparatus as claimed in claim 1, wherein the deployable catheter carrier comprises an expandable component that can be delivered to the desired position in a collapsed state in the sheath before emerging from the sheath to expand to hold its position within the main artery by contacting the arterial wall.

5. Apparatus as claimed in claim 4, wherein the expandable component is a cage.

6. Apparatus as claimed in claim 5, wherein the cage comprises a plurality of wires extending between the nose cone and a component axially movable relative to the nose cone.

7. Apparatus as claimed in claim 6, wherein the nose cone is on a rod and the axially movable component is a sheath on the rod.

8. Apparatus as claimed in claim 6, wherein when the axially movable component is moved away from the nose cone, the wires of the cage straighten so that the cage becomes narrow for deployment within the outer sheath and when the axially movable component is moved toward the nose cone the wires of the cam bend outwardly, such that the cane takes a bulbous form once the outer sheath has been withdrawn, which can grip against the artery wall to hold the cage in position.

9. Apparatus as claimed in claim 1, wherein the catheter is associated with a radio-opaque component whereby the position of the catheter carrier can be detected.

10. Apparatus as claimed in claim 9, wherein the catheter is linked to the cage by means of the radio-opaque component.

11. Apparatus as claimed in claim 9, wherein the radio-opaque component is a ring.

12. Apparatus as claimed in claim 11, wherein the ring is fixed to wires of the cage.

13. Apparatus as claimed in claim 12, wherein the ring has inwards spokes to which the end of the catheter is joined.

14. Apparatus as claimed in claim 1, wherein the catheter is sufficiently flexible to bend but sufficiently rigid so as not to deform too much.

15. A method of positioning a catheter relative to a side artery from a main artery comprising moving delivery means through the main artery to a desired position, the delivery means carrying a deployable catheter carrier having means for holding the catheter in a desired position within the main artery when delivered and means for detecting the position of the catheter carrier.

16. (canceled)
